(19) United States
(12) Patent Application Publication
(10) Pub. No.: US 2002/0133852 A1
(43) Pub. Date: Sep. 19, 2002

(54) Soybean SSRs and methods of genotyping

(76) Inventors: Brian M. Hauge, Beverly, MA (US); Roger J. Effertz, St. Louis, MO (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 09/969,373

(22) Filed: Oct. 2, 2001

Related U.S. Application Data
(63) Continuation-in-part of application No. 09/754,853, filed on Jan. 5, 2001.
(63) Continuation-in-part of application No. 09/760,427, filed on Jan. 13, 2001, now abandoned.
(63) Continuation-in-part of application No. 09/855,768, filed on May 15, 2001.
(60) Provisional application No. 60/174,880, filed on Jan. 7, 2000.

(51) Int. Cl. C12Q 1/68; G06F 19/00; G01N 33/48; G01N 33/50; A01H 5/00; C07H 21/04

(52) U.S. Cl. 800/312; 435/6; 702/20; 536/23.2

(57) ABSTRACT

SSR-containing soybean DNA loci useful for genotyping between at least two varieties of soybean. Sequences of the loci are useful for designing primers and probe oligonucleotides for detecting SSR polymorphisms in soybean DNA. SSR polymorphisms are useful for genotyping applications in soybean. The SSR-containing loci are useful to establish marker/trait associations, e.g. in linkage disequilibrium mapping and association studies, positional cloning and transgenic applications, marker-aided breeding and marker-assisted selection, hybrid prediction and identity by descent studies. The SSR markers are also useful in mapping libraries of DNA clones, e.g. for soybean QTLs and genes linked to SSR polymorphisms.

### CROSS-REFERENCE TO RELATED APPLICATIONS

This application is a continuation in part and claims priority under 35 U.S.C. §120 of U.S. applications Ser. No. 09/754,853 filed Jan. 5, 2001 (which claims priority to provisional application No. 60/174,880 filed Jan. 7, 2000), Ser. No. 09/760,427 filed Jan. 13, 2001, and Ser. No. 09/855,768 filed May 15, 2001, the disclosures of which are incorporated herein by reference in their entireties.

### INCORPORATION OF SEQUENCE LISTING

Two copies of the sequence listing (Copy 1 and Copy 2) and a computer readable form (CRF) of the sequence listing, all on CD-ROMs, each containing the file named pa_{—}00362.rpt, which is 938 kilobytes (measured in MS-Windows NT) and was created on 09-28-2001 are herein incorporated by reference.

### INCORPORATION OF TABLE

Two copies of Table 1 on CD-ROMs, each containing the file named pa_{—}00362.txt, which is 181 kilobytes (measured in MS-Windows NT) and was created on 09-28-2001, are herein incorporated by reference.

### FIELD OF THE INVENTION

Disclosed herein are simple sequence repeats (SSRs) of nucleic acids in soybean genomic DNA, nucleic acid molecules associated with such SSRs and methods of using such SSRs and molecules, e.g. in genotyping. Polymorphic SSRs are usefully associated with a variety of genes and QTLs including SCN resistance on linkage groups A2 and G.

### BACKGROUND

Polymorphisms are useful as genetic markers for genotyping applications in the agriculture field, e.g. in plant genetic studies and commercial breeding. See for instance U.S. Pat. Nos. 5,385,835; 5,437,697; 5,385,835; 5,492,547; 5,746,023; 5,962,764; 5,981,832; 6,100,030 and 6,219,964, the disclosures of all of which are incorporated herein by reference. The highly conserved nature of DNA combined with stable polymorphisms provide genetic markers which are both predictable and discerning of different genotypes. Among the classes of existing genetic markers are a variety of polymorphisms indicating genetic variation including restriction-fragment-length polymorphisms (RFLPs), amplified fragment-length polymorphisms (AFLPs), microsatellite or simple sequence repeats, single nucleotide polymorphisms (SNPs) and insertion/deletion polymorphisms (Indels). SSR markers are single locus markers with multiple alleles; and they are presently the DNA marker of choice in soybean marker applications because of their simplicity and their many alleles, which enables detection of polymorphism among elite cultivars and breeding lines. SSRs are well distributed throughout the soybean genome and have been used to distinguish the genotype of soybean cultivars and elite breeding lines. These methods have been developed for soybean and are well known in the field of molecular plant breeding (Rongwen, Theor. Appl. Gen. 90:43-48 (1995); Akkaya, Crop Sci. 35:1439-1445 (1995); Mansur, Crop Sci. 36:1327-1336 (1996); Diwan, Theor. Appl. Gen. 95:723-733 (1997); Simple sequence repeat DNA marker analysis, in “DNA markers: Protocols, applications, and overviews: (1997) 173-185, Cregan, et al., eds., Wiley-Liss N.Y.; all of which is herein incorporated by reference in its' entirely. In a particularly preferred embodiment, a marker molecule is detected by SSR techniques. It is understood that SSR primers can hybridize to a combination of plant DNA and adapter DNA (e.g. EcoRI adapter or MseI adapter, Vos et al., Nucleic Acids Res. 23:4407-4414 (1995)).

Technological advances during the past 75 years have enabled U.S. soybean producers to more than triple average soybean yield from 12 bushels per acre in 1924 to nearly 40 bushels per acre in recent years. A substantial part of the yield increase is attributed to genetic improvement through breeding. Because the number of genetic markers for soybean is limited, the discovery of additional genetic markers will facilitate improvements from marker-assisted breeding and other genotyping applications such as marker-trait association studies, gene mapping, gene discovery and marker-assisted selection. A limited number of soybean markers are available. Reference is made to the SoyBase web site maintained by the USDA and Iowa State University at “macgrant.agron.iastate.edu” which provides soybean loci for morphological, biochemical and molecular markers including soybean SSR markers and PCR primers used to amplify SSR loci. However, to obtain adequate genome wide coverage for quantitative trait loci (QTL) analysis and marker assisted breeding applications, development of additional SSRs is desirable.

### SUMMARY OF THE INVENTION

This invention provides a large number of SSRs which can be useful as genetic markers for soybean. These genetic markers comprise soybean DNA loci which are useful for genotyping applications. A soybean locus of this invention comprises at least 15, more preferably at least 18, even more preferably at least 20, consecutive nucleotides adjacent to an SSR. Table 1 provides a list of 1531 SSR-containing loci in selected regions of soybean linkage groups A1, G, A2, H and M including the previously-known SSR markers Satt315, Satt632, Sat_{—}162 and Satt424 on linkage group A2 and Satt275, Satt163, Sat_{—}168, Satt309, Sat_{—}141, Sat_{—}163, Satt610, Satt570 and Satt235 on linkage group G. More particularly, a soybean locus of this invention has a nucleic acid sequence which is at least 90%, preferably at least 95%, identical to the sequence of the same number of nucleotides in either strand of a segment of soybean DNA which is adjacent to the SSR.

In one aspect of the invention the soybean loci are provided in one or more data sets of DNA sequences, i.e. data sets comprising up to a finite number of distinct sequences of SSR containing loci. The finite number of SSR loci in a data set can be as few as 2 or up to 1000 or more, e.g. at least 5, 10, 25, 40, 75, 100 or 500 loci. Such data sets are useful for genotyping applications of a large scale or involving large numbers of plants. In a useful aspect of the invention the data set of soybean SSR loci is recorded on a computer readable medium.

In another aspect of the invention the SSRs in the loci of the invention are mapped onto the soybean genome, e.g. as a genetic map of the soybean genome comprising map positions of SSRs, as illustrated in FIGS. 1 and 2, or as a physical map of SSR positions as indicated in Table 1 for the SSR-containing loci of SEQ ID NO:1 through SEQ ID NO:1531. The genetic linkage data can also be recorded on computer readable medium. Preferred embodiments of the invention provide genetic maps of SSRs at high densities across a map of a region of soybean genome. Especially useful genetic maps comprise SSR markers at an average distance of not more than 10 centiMorgans (cM) on a linkage group, e.g. not more than 5 cM, more preferably at an average distance between markers of not more than 2 cM, e.g. not more than 1 cM, even more preferably at an average distance between markers of not more than 0.5 cM in a region of a soybean genome.

This invention also provides nucleic acid molecules for identifying SSR polymorphisms; such molecules are preferably oligonucleotides which are useful as PCR primers for amplifying a segment of a soybean genome, e.g. a polymorphic locus, and hybridization probes for use in assays to identify in soybean DNA the presence or absence of particular polymorphisms. Nucleic acid molecules useful as PCR primers are typically provided in pairs for the amplification of a segment of soybean DNA comprising at least one polymorphism, where each molecule comprises at least 12, more usually at least 15, nucleotide bases. The nucleotide sequence of one of the primer molecules is preferably at least 90 percent identical to a sequence of the same number of consecutive nucleotides in one strand of a segment of soybean DNA in a polymorphic locus and the sequence of the other of the primer molecules is at least 90 percent identical to a sequence of the same number of consecutive nucleotides in the other strand of said segment of soybean DNA in the polymorphic locus. In addition to the designed complementary sequence a primer can have tags, e.g. a polynucleotide sequence useful for analytical assay at the 5′ end of the primer. Preferably the primers are capable of hybridizing under high stringency conditions to the strands of DNA in the polymorphic locus. Preferably such primers are provided and used in pairs which flank at least one polymorphism in the segment of soybean DNA in a polymorphic locus. Reference is made to the SEQ ID No: 1532 through SEQ ID NO:4593 in Table 1 which identifies nucleic acid sequences for forward and reverse primers for the SSR-containing loci of SEQ ID NO: 1 through SEQ ID NO: 1531.

This invention also provides methods of using the loci and polymorphism of this invention, e.g. in genotyping and related applications. One aspect. of this invention provides methods of finding polymorphisms in soybean DNA by comparing DNA sequence in at least two soybean lines where the sequence is selected by using oligonucleotide primers which are designed to amplify the polymorphic soybean DNA locus.

This invention also provides methods of genotyping by assaying DNA or mRNA from tissue of at least one soybean line to identify the presence of an SSR polymorphism linked to a polymorphic locus of this invention. In preferred aspects of the invention genotyping uses an SSR polymorphism identified in soybean linkage group A2 or G. In another preferred aspect of the invention genotyping comprises identifying one or more phenotypic traits for at least two soybean lines and determining associations between traits and polymorphisms, e.g. lines with complementary traits are identified and selected for breeding to improve heterosis. Assays for such genotyping can employ sufficient nucleic acid molecules to identify the presence of at least 2 and up to 5000 or more distinct polymorphisms, e.g. where the number of distinct polymorphisms is at least 5, 10, 25, 40, 75, 100, 500, 1000, 2000, 3000 or 4000.

This invention also provides methods of investigating a soybean allele by determining the presence of a polymorphism in the nucleic acid sequence of nucleic acid molecules isolated from one or more soybean plants where the polymorphism is linked to a polymorphic locus of the invention.

This invention also provides methods of mapping soybean genomic sequence by identifying the presence of a mapped polymorphism in the genomic sequence where the mapped polymorphism is linked to a polymorphic locus of the invention, e.g. a mapped polymorphism on a genetic map of this invention.

This invention also provides methods of breeding soybean by selecting a soybean line having a polymorphism associated by linkage disequilibrium to a trait of interest where the polymorphism is linked to a polymorphic locus of the invention.

This invention also provides methods of associating a phenotype to a genotype in soybean plants by identifying a set of one or more distinct phenotypic traits characterizing the soybean plants. DNA or mRNA in tissue from at least two soybean plants having allelic DNA is assayed to identify the presence or absence of a set of distinct SSR polymorphisms. Associations between the set of SSR polymorphisms and set of phenotypic traits are identified where the set of SSR polymorphisms comprises at least one, more preferably at least 10, SSR-containing locus of the invention, e.g. at least 10 SSR polymorphisms linked to mapped SSR-containing loci of this invention. In a more preferred aspect traits are associated to genotypes in a segregating population of soybean plants having allelic DNA in loci of a chromosome which confers a phenotypic effect on a trait of interest and where a polymorphism is located in such loci and where the degree of association among the polymorphisms and between the polymorphisms and the traits permits determination of a linear order of the polymorphism and the trait loci. In such methods polymorphisms are linked to loci permitting disequilibrium mapping of the loci.

This invention also provides methods of identifying genes associated with a trait of interest by identifying linkage of at least one polymorphism to a trait of interest where the polymorphism is linked to a polymorphic locus of the invention, identifying a genomic clone containing the locus and identifying genes linked to the locus. In preferred aspects of the invention such association is useful in marker assisted breeding an/or marker assisted selection.

This invention also provides methods to screen for traits by interrogating a collection of SSR polymorphisms at an average density of less than 10 cM on a genetic map of soybean. The presence or absence of an SSR polymorphism linked to a polymorphic locus of the invention is correlated such traits.

### BRIEF DESCRIPTION OF THE DRAWINGS

FIGS. 1 and 2 are cytogenetic, genetic and physical maps for regions of soybean linkage groups A2 and G comprising SSR-containing loci.

Definitions:

As used herein certain terms are defined as follows.

An “SSR” means a simple sequence repeat of DNA sequence.

An “allele” means an alternative sequence at a particular locus; the length of an allele can be as small as 1 nucleotide base, but is typically larger. Allelic sequence can be amino acid sequence or nucleic acid sequence. A “locus” is a short sequence that is usually unique and usually found at one particular location by a point of reference, e.g. a short DNA sequence that is a gene, or part of a gene or intergenic region. A locus of this invention comprises an SSR and an adjacent segment that is sufficiently long as to be amplified to a unique PCR product. The loci of this invention comprise an SSR that is a polymorphism in at least certain individuals. “Genotype” means the specification of an allelic composition at one or more loci within an individual organism. In the case of diploid organisms, there are two alleles at each locus; a diploid genotype is said to be homozygous when the alleles are the same, and heterozygous when the alleles are different.

“Phenotype” means the detectable characteristics of a cell or organism which are a manifestation of gene expression.

“Marker” mean polymorphic sequence. A “polymorphism” is a variation among individuals in sequence, particularly in DNA sequence. Useful polymorphisms of this invention include SSRs.

“Marker Assay” means an method for detecting a polymorphism at a particular locus using a particular method, e.g. phenotype (such as seed color, flower color, or other visually detectable trait), electrophoresis, e.g. gel electrophoresis such as Southern blots, of length polymorphisms associated with SSRs.

“Linkage Group” refers to a soybean chromosome. As the nomenclature of soybean linkage groups has been changing, the following table correlates linkage group nomenclature.

| Linkage Group | Linkage Group |
|---|---|
| Number Designation | Letter Designation |
| 1 | J |
| 2 | E |
| 3 | A2 |
| 4 | B1 |
| 5 | G |
| 6 | N |
| 7 | A1 |
| 8 | D1a + Q |
| 9 | C2 |
| 10 | H |
| 11 | M |
| 12 | D2 |
| 13 | F |
| 14 | L |
| 15 | I |
| 16 | D1b + W |
| 17 | O |
| 18 | C1 |
| 19 | K |
| 20 | B2 |

Reference to linkage groups in connection with describing the markers of this invention is by reference to the alphabetic designations, e.g. A2, G, A1, H and M.

“Linkage” refers to relative frequency at which types of gametes are produced in a cross. For example, if locus A has allele “A” or “a” and locus B has allele “B” or “b” and a cross between parent I with AABB and parent B with aabb will produce four possible gametes where the alleles are segregated into AB, Ab, aB and ab. The null expectation is that there will be independent equal segregation into each of the four possible genotypes, i.e. with no linkage ¼ of the gametes will of each genotype. Segregation of gametes into a genotypes differing from ¼ are attributed to linkage.

“Linkage disequilibrium” is defined in the context of the relative frequency of gamete types in a population of many individuals in a single generation. If the frequency of allele A is p, a is p′, B is q and b is q′, then the expected frequency (with no linkage disequilibrium) of genotype AB is pq, Ab is pq′, aB is p′q and alb is p′q′. Any deviation from the expected frequency is called linkage disequilibrium. Two loci are said to be “genetically linked” when they are in linkage disequilibrium

“Quantitative Trait Locus (QTL)” means a locus that controls to some degree numerically representable traits that are usually continuously distributed.

Nucleic acid molecules or fragments thereof of the present invention are capable of hybridizing to other nucleic acid molecules under certain circumstances. As used herein, two nucleic acid molecules are said to be capable of hybridizing to one another if the two molecules are capable of forming an anti-parallel, double-stranded nucleic acid structure. A nucleic acid molecule is said to be the “complement” of another nucleic acid molecule if they exhibit “complete complementarity” i.e. each nucleotide in one sequence is complementary to its base pairing partner nucleotide in another sequence. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Nucleic acid molecules which hybridize to other nucleic acid molecules, e.g. at least under low stringency conditions are said to be “hybridizable cognates” of the other nucleic acid molecules. Conventional stringency conditions are described by Sambrook et al., Molecular Cloning, A Laboratory Manual, 2nd Ed., Cold Spring Harbor Press, Cold Spring Harbor, N.Y. (1989) and by Haymes et al., Nucleic Acid Hybridization, A Practical Approach, IRL Press, Washington, D.C. (1985), each of which is incorporated herein by reference. Departures from complete complementarity are therefore permissible, as long as such departures do not completely preclude the capacity of the molecules to form a double-stranded structure. Thus, in order for a nucleic acid molecule to serve as a primer or probe it need only be sufficiently complementary in sequence to be able to form a stable double-stranded structure under the particular solvent and salt concentrations employed.

Appropriate stringency conditions which promote DNA hybridization, for example, 6.0× sodium chloride/sodium citrate (SSC) at about 45° C., followed by a wash of 2.0× SSC at 50° C., are known to those skilled in the art or can be found in Current Protocols in Molecular Biology, John Wiley & Sons, N.Y. (1989), 6.3.1-6.3.6, incorporated herein by reference. For example, the salt concentration in the wash step can be selected from a low stringency of about 2.0× SSC at 50° C. to a high stringency of about 0.2× SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

In a preferred embodiment, a nucleic acid molecule of the present invention will specifically hybridize to one strand of a segment of soybean DNA having a nucleic acid sequence as set forth in SEQ ID NO: 1 through SEQ ID NO: 1531 under moderately stringent conditions, for example at about 2.0× SSC and about 65° C., more preferably under high stringency conditions such as 0.2× SSC and about 65° C.

As used herein “sequence identity” refers to the extent to which two optimally aligned polynucleotide or peptide sequences are invariant throughout a window of alignment of components, e.g. nucleotides or amino acids. An “identity fraction” for aligned segments of a test sequence and a reference sequence is the number of identical components which are shared by the two aligned sequences divided by the total number of components in reference sequence segment, i.e. the entire reference sequence or a smaller defined part of the reference sequence. “Percent identity” is the identity fraction times 100.

### DETAILED DESCRIPTION OF PREFERRED EMBODIMENTS

A. Nucleic Acid Molecules—Loci, Primers and Probes

The SSR-containing loci identified by SEQ ID NO: 1 through SEQ ID NO: 1531 in Table 1 represent soybean DNA loci having SSRs which are useful as markers for genotyping between two or more varieties of soybean. The 1531 SSR-containing loci in Table 1 are found in selected regions of linkages groups A2, G, A1, H and M and include the markers of this invention and previously-known SSR markers, i.e. Satt315 (SEQ ID NO:8), Satt632 (SEQ ID NO: 58), Sat_{—}162 (SEQ ID NO:182) and Satt424 (SEQ ID NO:472) on linkage group A2 and Satt275 (SEQ ID NO:560), Satt163 (SEQ ID NO:658), Sat_{—}168 (SEQ ID NO:798), Satt309 (SEQ ID NO:819), Sat_{—}141 (SEQ ID NO: 1063), Sat_{—}163 (SEQ ID NO:1065), Satt610 (SEQ ID NO:1181), Satt570 (SEQ ID NO:1249) and Satt235 (SEQ ID NO:1303) on linkage group G.

Each SSR-containing soybean DNA locus comprises an SSR flanked by reference polynucleotides of at least 15, more preferably at least 18, even more preferably at least 20, consecutive nucleotides. An SSR marker can be characterized by the DNA sequence of either, preferably both, of the flanking polynucleotides or the complements thereof. Such SSR-containing soybean DNA loci are more particularly characterized as being at least 90%, more preferably at least 95%, identical to the sequence of a reference polynucleotide the same number of consecutive nucleotides which are adjacent to an S SR identified in Table 1 or the complement of such reference polynucleotide. More preferably for some alleles, such SSR soybean DNA loci have a nucleic acid sequence having at least 98%, or in some cases at least 99%, sequence identity, to the sequence of the same number of nucleotides in either strand of a segment of soybean DNA which is adjacent to the SSR. The nucleotide sequence of one strand of such a segment of soybean DNA may be found in a sequence in the group consisting of SEQ ID NO: 1 through SEQ ID NO: 1531.

It is understood by the very nature of SSRs that for at least some loci there may be no SSR polymorphism, per se, between any two lines of soybean. Thus, sequence identity can be determined for sequence that is exclusive of the polymorphism sequence. Because of duplication in the soybean genome it is understood that certain SSRs can be represented at multiple loci on the same or a different linkage group; see, for instance, the loci of SEQ ID NO:228 and SEQ ID NO:297 on linkage group A2 which are duplicates of an SSR having 7 repeating units of TG. Another duplicated SSR is in the loci of SEQ ID NO: 154 and DEQ ID NO: 1403. The primer picking software selected one common primer for both loci, i.e. reverse primer of SEQ ID NO: 1839 is identical to forward primer of SEQ ID NO:4336. The other primers are different. In Table 1 the repeat unit for SEQ ID NO: 1403 is reported as CATT and for SEQ ID NO: 154 as ATGA. However, inspection of the amplicons of DEQ ID NO: 154 and SEQ ID NO: 1403 shows that the repeat units are complements with a shift in sequence.

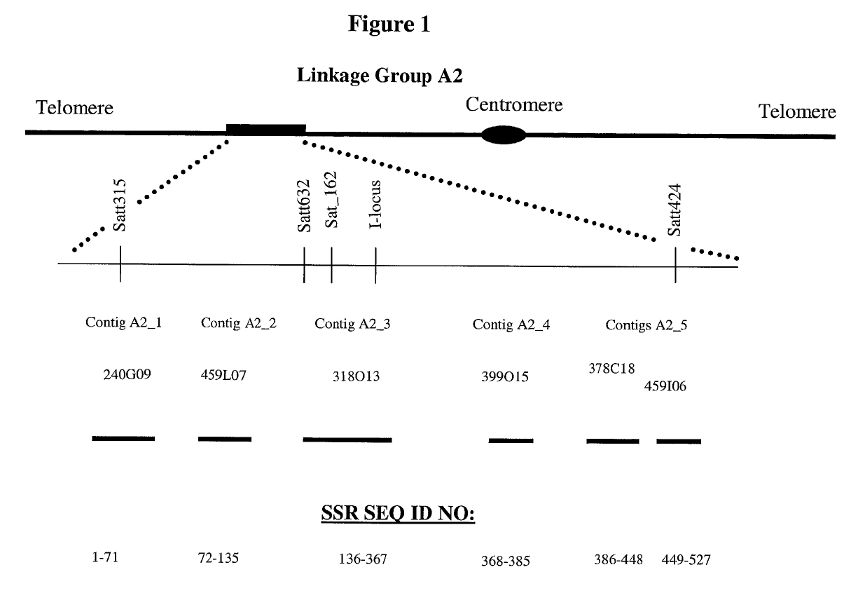

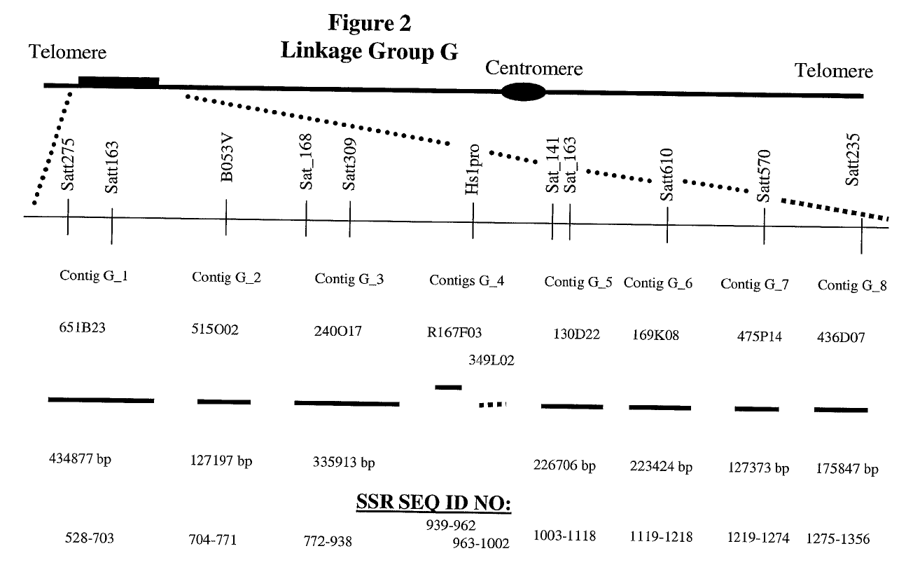

The data associated with the SSRs-containing loci in Table enables the construction of a physical map of SSR markers in the segments of a linkage group; see, for instance, FIGS. 1 and 2. For many genotyping applications it is useful to employ as markers polymorphisms from more than one locus. Thus, one aspect of the invention provides a collection of different loci. The number of loci in such a collection can vary but will be a finite number, e.g. as few as 2 or 5 or 10 or 25 loci or more, for instance up to 40 or 75 or 100 or more loci.

Another aspect of the invention provides nucleic acid molecules which are capable of hybridizing to the polymorphic soybean loci of this invention. In certain embodiments of the invention, e.g. which provide PCR primers, such molecules comprises at least 15 nucleotide bases, more preferably between 18 and 24 nucleotide bases, generally not more than 30 nucleotide bases. Molecules useful as primers can hybridize under high stringency conditions to a one of the strands of a segment of DNA in a polymorphic locus of this invention. Primers for amplifying DNA are provided in pairs, i.e. a forward primer and a reverse primer. One primer will be complementary to one strand of DNA in the locus and the other primer will be complementary to the other strand of DNA in the locus, i.e. the sequence of a primer is preferably at least 90%, more preferably at least 95%, identical to a sequence of the same number of nucleotides in one of the strands. It is understood that such primers can hybridize to sequence in the locus which is distant from the SSR, e.g. at least 5, 10, 20, 50 or up to about 100 or more nucleotide bases away from the polymorphism. Design of a primer of this invention will depend on factors well known in the art, e.g. avoidance or repetitive sequence. Exemplary primers include the oligonucleotides of SEQ ID NO: 1532 through SEQ ID NO:4593, which are listed in Table 1 in pairs in order of the SSR markers and identified by a Seq ID corresponding to the Seq ID of the corresponding SSR marker followed by “fw” or “rv”, indicating the forward and reverse primers respectively. The sequence numbers for a primer pair for a particular SSR-containing locus, e.g. SEQ ID NO:(n), are determined as SEQ ID NO:(1531+2n−1) for the forward primer and SEQ ID NO:(1531+2n) for the reverse primer.

B. Identifying Polymorphisms

Polymorphisms in a genome can be determined by comparing cDNA sequence from different lines. While the detection of polymorphisms by comparing cDNA sequence is relatively convenient, evaluation of cDNA sequence allows no information about the position of introns in the corresponding genomic DNA. Moreover, polymorphisms in non-coding sequence cannot be identified from cDNA. This can be a disadvantage, e.g. when using cDNA-derived polymorphisms as markers for genotyping of genomic DNA. More efficient genotyping assays can be designed if the scope of polymorphisms includes those present in non-coding unique sequence.

Genomic DNA sequence is more useful than cDNA for identifying and detecting SSR polymorphisms. SSR polymorphisms in a genome can be determined by analyzing segments of genomic sequence for repetitive patterns, such as AT, TTA, AGTA, ATTT, and other patterns illustrated in Table 1. Sequence analyzing algorithms are a convenient way to identify SRR polymorphisms.

C. Detecting Polymorphisms

Polymorphisms in DNA sequences can be detected by a variety of effective methods well known in the art including those disclosed in U.S. Pat. No. 5,468,610; 5,766,847 and 6,090,558; all of which are incorporated herein by reference in their entireties. Repeat polymorphisms can be analyzed by PCR amplifying a segment containing the polymorphism and resolving the amplified segments using gel electrophoresis to differentiate SSRs of different lengths, e.g. by comparing separated individual bands on an electrophoresis gel or by autoradiography. SSRs can also be detected by mass spectroscopy methods as disclosed in U.S. Pat. No. 6,090,558; an advantages of using mass spectrometry include a dramatic increase in both the speed of analysis (a few seconds per sample) and the accuracy of direct mass measurements.

D. Use of Polymorphisms to Establish Marker/Trait Associations

The polymorphisms in the loci of this invention can be used in marker/trait associations which are inferred from statistical analysis of genotypes and phenotypes of the members of a population. These members may be individual organisms, e.g. soybean, families of closely related individuals, inbred lines, dihaploids or other groups of closely related individuals. Such soy groups are referred to as “lines”, indicating line of descent. The population may be descended from a single cross between two individuals or two lines (e.g. a mapping population) or it may consist of individuals with many lines of descent. Each individual or line is characterized by a single or average trait phenotype and by the genotypes at one or more marker loci.

Several types of statistical analysis can be used to infer marker/trait association from the phenotype/genotype data, but a basic idea is to detect markers, i.e. polymorphisms, for which alternative genotypes have significantly different average phenotypes. For example, if a given marker locus A has three alternative genotypes (AA, Aa and aa), and if those three classes of individuals have significantly different phenotypes, then one infers that locus A is associated with the trait. The significance of differences in phenotype may be tested by several types of standard statistical tests such as linear regression of marker genotypes on phenotype or analysis of variance (ANOVA). Commercially available, statistical software packages commonly used to do this type of analysis include SAS Enterprise Miner (SAS Institute Inc., Cary, N.C.) and Splus (Insightful Corporation. Cambridge, Mass.). When many markers are tested simultaneously, an adjustment such as Bonferonni correction is made in the level of significance required to declare an association.

Often the goal of an association study is not simply to detect marker/trait associations, but to estimate the location of genes affecting the trait directly (i.e. QTLs) relative to the marker locations. In a simple approach to this goal, one makes a comparison among marker loci of the magnitude of difference among alternative genotypes or the level of significance of that difference. Trait genes are inferred to be located nearest the marker(s) that have the greatest associated genotypic difference. In a more complex analysis, such as interval mapping (Lander and Botstein, Genetics 121:185-199 (1989)), each of many positions along the genetic map (say at 1 cM intervals) is tested for the likelihood that a QTL is located at that position. The genotype/phenotype data are used to calculate for each test position a LOD score (log of likelihood ratio). When the LOD score exceeds a critical threshold value, there is significant evidence for the location of a QTL at that position on the genetic map (which will fall between two particular marker loci).

a. Linkage Disequilibrium Mapping and Association Studies

Another approach to determining trait gene location is to analyze trait-marker associations in a population within which individuals differ at both trait and marker loci. Certain marker alleles may be associated with certain trait locus alleles in this population due to population genetic process such as the unique origin of mutations, founder events, random drift and population structure. This association is referred to as linkage disequilibrium. In linkage disequilibrium mapping, one compares the trait values of individuals with different genotypes at a marker locus. Typically, a significant trait difference indicates close proximity between marker locus and one or more trait loci. If the marker density is appropriately high and the linkage disequilibrium occurs only between very closely linked sites on a chromosome, the location of trait loci can be very precise.

A specific type of linkage disequilibrium mapping is known as association studies. This approach makes use of markers within candidate genes, which are genes that are thought to be functionally involved in development of the trait because of information such as biochemistry, physiology, transcriptional profiling and reverse genetic experiments in model organisms. In association studies, markers within candidate genes are tested for association with trait variation. If linkage disequilibrium in the study population is restricted to very closely linked sites (i.e. within a gene or between adjacent genes), a positive association provides nearly conclusive evidence that the candidate gene is a trait gene.

b. Positional Cloning and Transgenic Applications

Traditional linkage mapping typically localizes a trait gene to an interval between two genetic markers (referred to as flanking markers). When this interval is relatively small (say less than 1 Mb), it becomes feasible to precisely identify the trait gene by a positional cloning procedure. A high marker density is required to narrow down the interval length sufficiently. This procedure requires a library of large insert genomic clones (such as a BAC library), where the inserts are pieces (usually 100-150 kb in length) of genomic DNA from the species of interest. The library is screened by probe hybridization or PCR to identify clones that contain the flanking marker sequences. Then a series of partially overlapping clones that connects the two flanking clones (a “contig”) is built up through physical mapping procedures. These procedures include fingerprinting, STS content mapping and sequence-tagged connector methodologies. Once the physical contig is constructed and sequenced, the sequence is searched for all transcriptional units. The transcriptional unit that corresponds to the trait gene can be determined by comparing sequences between mutant and wild type strains, by additional fine-scale genetic mapping, and/or by functional testing through plant transformation. Trait genes identified in this way become leads for transgenic product development. Similarly, trait genes identified by association studies with candidate genes become leads for transgenic product development.

c. Marker-Aided Breeding and Marker-Assisted Selection

When a trait gene has been localized in the vicinity of genetic markers, those markers can be used to select for improved values of the trait without the need for phenotypic analysis at each cycle of selection. In marker aided breeding and marker-assisted selection, associations between trait genes and markers are established initially through genetic mapping analysis (as in A.1 or A.2). In the same process, one determines which marker alleles are linked to favorable trait gene alleles. Subsequently, marker alleles associated with favorable trait gene alleles are selected in the population. This procedure will improve the value of the trait provided that there is sufficiently close linkage between markers and trait genes. The degree of linkage required depends upon the number of generations of selection because, at each generation, there is opportunity for breakdown of the association through recombination.

Prediction of Crosses for New Inbred Line Development

The associations between specific marker alleles and favorable trait gene alleles also can be used to predict what types of progeny may segregate from a given cross. This prediction may allow selection of appropriate parents to generation populations from which new combinations of favorable trait gene alleles are assembled to produce a new inbred line. For example, if line A has marker alleles previously known to be associated with favorable trait alleles at loci 1, 20 and 31, while line B has marker alleles associated with favorable effects at loci 15, 27 and 29, then a new line could be developed by crossing A×B and selecting progeny that have favorable alleles at all 6 trait loci.

E. Use of Polymorphism Assay for Mapping a Library of DNA Clones

The polymorphisms and loci of this invention are useful for identifying and mapping DNA sequence of QTLs and genes linked to the polymorphisms. For instance, BAC or YAC clone libraries can be queried using polymorphisms linked to a trait to find a clone containing specific QTLs and genes associated with the trait. For instance, QTLs and genes in a plurality, e.g. hundreds or thousands, of large, multi-gene sequences can be identified by PCR amplification with oligonucleotide primers which anneal to a mapped and/or linked polymorphism. Such PCR screening can be improved by providing clone sequence in a high density array. The screening method is more preferably enhanced by employing a pooling strategy to significantly reduce the number of PCR reactions required to identify a clone containing the polymorphism. When the polymorphisms are mapped, the screening effectively maps the clones.

For instance, in a case where thousands of clones are arranged in a defined array, e.g. in 96 well plates, the plates can be arbitrarily arranged in three-dimensionally, arrayed stacks of wells each comprising a unique DNA clone. The wells in each stack can be represented as discrete elements in a three dimensional array of rows, columns and plates. In one aspect of the invention the number of stacks and plates in a stack are about equal to minimize the number of assays. The stacks of plates allow the construction of pools of cloned DNA.

For a three-dimensionally arrayed stack pools of cloned DNA can be created for (a) all of the elements in each row, (b) all of the elements of each column, and (c) all of the elements of each plate. PCR screening of the pools with an oligonucleotide primer which anneals to an SSR locus unique to one of the clones will provide a positive indication for one column pool, one row pool and one plate pool, thereby indicating the well element containing the target clone.

In the case of multiple stacks, additional pools of all of the clone DNA in each stack allows indication of the stack having the row-column-plate coordinates of the target clone. For instance, a 4608 clone set can be disposed in 48 96-well plates. The 48 plates can be arranged in 8 sets of 6 plate stacks providing 6×12×8 three-dimensional arrays of elements, i.e. each stack comprises 6 stacks of 8 rows and 12 columns. For the entire clone set there are 36 pools, i.e. 6 stack pools, 8 row pools, 12 column pools and 8 stack pools. Thus, a maximum of 36 PCR reactions is required to find the clone harboring QTLs or genes associated or linked to each mapped polymorphism.

Once a clone is identified, oligonucleotide primers designed from the locus of the SSR can be used for positional cloning of the linked QTL and/or genes.

F. Computer Readable Media and Databases

The sequences of nucleic acid molecules of this invention can be “provided” in a variety of mediums to facilitate use, e.g. a database or computer readable medium, which can also contain descriptive annotations in a form that allows a skilled artisan to examine or query the sequences and obtain useful information. In one embodiment of the invention computer readable media may be prepared that comprise nucleic acid sequences where at least 10% or more, e.g. at least 25%, or even at least 50% or more of the sequences of the loci and nucleic acid molecules of this invention. For instance, such database or computer readable medium may comprise sets of the loci of this invention or sets of primers and probes useful for assaying the polymorphisms of this invention. In addition such database or computer readable medium may comprise a figure or table of the mapped or unmapped polymorphisms or this invention and genetic maps.

As used herein “database” refers to any representation of retrievable collected data including computer files such as text files, database files, spreadsheet files and image files, printed tabulations and graphical representations and combinations of digital and image data collections. In a preferred aspect of the invention, “database” means a memory system that can store computer searchable information. Currently, preferred database applications include those provided by DB2, Sybase and Oracle.

As used herein, “computer readable media” refers to any medium that can be read and accessed directly by a computer. Such media include, but are not limited to: magnetic storage media, such as floppy discs, hard disc, storage medium and magnetic tape; optical storage media such as CD-ROM; electrical storage media such as RAM and ROM; and hybrids of these categories such as magnetic/optical storage media. A skilled artisan can readily appreciate how any of the presently known computer readable mediums can be used to create a manufacture comprising computer readable medium having recorded thereon a nucleotide sequence of the present invention.

As used herein, “recorded” refers to the result of a process for storing information in a retrievable database or computer readable medium. For instance, a skilled artisan can readily adopt any of the presently known methods for recording information on computer readable medium to generate media comprising the mapped polymorphisms and other nucleotide sequence information of the present invention. A variety of data storage structures are available to a skilled artisan for creating a computer readable medium where the choice of the data storage structure will generally be based on the means chosen to access the stored information. In addition, a variety of data processor programs and formats can be used to store the polymorphisms and nucleotide sequence information of the present invention on computer readable medium.

Computer software is publicly available which allows a skilled artisan to access sequence information provided in a computer readable medium. The examples which follow demonstrate how software which implements a search algorithm such as the BLAST algorithm (Altschul et al., J. Mol. Biol. 215:403-410 (1990), incorporated herein by reference) and the BLAZE algorithm (Brutlag et al., Comp. Chem. 17:203-207 (1993), incorporated herein by reference) on a Sybase system can be used to identify DNA sequence which is homologous to the sequence of loci of this invention with a high level of identity. Sequence of high identity can be compared to find polymorphic markers useful with a maize varieties.

The present invention further provides systems, particularly computer-based systems, which contain the sequence information described herein. Such systems are designed to identify commercially important sequence segments of the nucleic acid molecules of this invention. As used herein, “a computer-based system” refers to the hardware, software and memory used to analyze the nucleotide sequence information. A skilled artisan can readily appreciate that any one of the currently available computer-based system are suitable for use in the present invention.

As indicated above, the computer-based systems of the present invention comprise a database having stored therein polymorphic markers, genetic maps, and/or the sequence of nucleic acid molecules of the present invention and the necessary hardware and software for supporting and implementing genotyping applications.

### EXAMPLE 1

This example illustrates the determination of soybean genomic DNA sequence from BAC clones of the soybean line A3244. Two basic methods can be used for DNA sequencing, the chain termination method of Sanger et al., Proc. Natl. Acad. Sci. USA 74:5463-5467 (1977) and the chemical degradation method of Maxam and Gilbert, Proc. Natl. Acad. Sci. USA 74:560-564 (1977). Automation and advances in technology such as the replacement of radioisotopes with fluorescence-based sequencing have reduced the effort required to sequence DNA (Craxton, Methods, 2:20-26 (1991), Ju et al., Proc. Natl. Acad. Sci. USA 92:4347-4351 (1995) and Tabor and Richardson, Proc. Natl. Acad. Sci. USA 92:6339-6343 (1995). Automated sequencers are available from, for example, Applied Biosystems, Foster City, Calif. (ABI Prism® systems); Pharmacia Biotech, Inc., Piscataway, N.J. (Pharmacia ALF), LI-COR, Inc., Lincoln, Nebr. (LI-COR 4,000) and Millipore, Bedford, Mass. (Millipore BaseStation).

In addition, advances in capillary gel electrophoresis have also reduced the effort required to sequence DNA and such advances provide a rapid high resolution approach for sequencing DNA samples (Swerdlow and Gesteland, Nucleic Acids Res. 18:1415-1419 (1990); Smith, Nature 349:812-813 (1991); Luckey et al., Methods Enzymol. 218:154-172 (1993); Lu et al., J. Chromatog. A. 680:497-501 (1994); Carson et al., Anal. Chem. 65:3219-3226 (1993); Huang et al., Anal. Chem. 64:2149-2154 (1992); Kheterpal et al., Electrophoresis 17:1852-1859 (1996); Quesada and Zhang, Electrophoresis 17:1841-1851 (1996); Baba, Yakugaku Zasshi 117:265-281 (1997).

A number of sequencing techniques are known in the art, including fluorescence-based sequencing methodologies. These methods have the detection, automation and instrumentation capability necessary for the analysis of large volumes of sequence data. An ABI Prism®377 DNA Sequencer (Applied Biosystems, Foster City, Calif.) allows rapid electrophoresis and data collection. With these types of automated systems, fluorescent dye-labeled sequence reaction products are detected and data entered directly into the computer, producing a chromatogram that is subsequently viewed, stored, and analyzed using the corresponding software programs. These methods are known to those of skill in the art and have been described and reviewed (Birren et al., Genome Analysis: Analyzing DNA,1, Cold Spring Harbor, N.Y. (1999).

Sequence base calling from trace files and quality scores are assigned by PHRED which is available from CodonCode Corporation, Dedham, Mass. and is described by Brent Ewing, et al. “Base-calling of automated sequencer traces using phred”, 1998, Genome Research, Vol. 8, pages 175-185 and 186-194, incorporated herein by reference.

After the base calling is completed, sequence quality is improved by cutting poor quality end sequence. If the resulting sequence is less than 50 bp, it is deleted. Sequence with an overall quality of less than 12.5 is deleted. And, contaminating sequence, e.g. E. coli BAC and vector sequences and sub-cloning vector, are removed. Contigs are assembled using Pangea Clustering and Alignment Tools which is available from DoubleTwist Inc., Oakland, Calif. by comparing pairs of sequences for overlapping bases. The overlap is determined using the following high stringency parameters: word size=8; window size=60; and identity is 93%. The clusters are reassembled using PHRAP fragment assembly program which is available from CodonCode Corporation using a “repeat stringency” parameter of 0.5 or lower. The final assembly output contains a collection of sequences including contig sequences which represent the consensus sequence of overlapping clustered sequences (contigs) and singleton sequences which are not present in any cluster of related sequences (singletons). Collectively, the contigs and singletons resulting from a DNA assembly are referred to as islands.

### EXAMPLE 2

This example illustrates SSRs which have been identified by searching for repeat segments of sequences in the contigs and singletons of genomic sequence of the soybean line A3244 as prepared as in Example 1. A plurality of loci having SSRs are reported as SEQ ID NO: 1 through SEQ ID NO: 1531 and identified more particularly in Table 1 which identifies the repeat unit and physical mapping distance between SSRs in soybean line A3244. The style of Table 1 is illustrated by reference to the following Abbreviated Table 1

Abbreviated Table 1

| | | | | Dis- |
|---|---|---|---|---|
| | | | | tance |
| | | | | from |
| | | | | Prev- |
| Seq | | Repeat | Repeat | ious |
| Num | Seq ID | Unit | Times | Marker |
| 1 | 240G09_region_A2_1_164_14 | TCG | 5 | _{—} |
| 2 | 240G09_region_A2_1_1076_51 | AT | 26 | 912 |
| 3 | 240G09_region_A2_1_3791_12 | TTC | 4 | 2715 |
| 1531 | 344J16_region_M_1_8102_14 | AATA | 4 | 2245 |
| 1532 | 240G09_region_A2_1_164_14_{—} | | | |
| | fw | | | |
| 1533 | 240G09_region_A2_1_164_14_{—} | | | |
| | rv | | | |
| 4592 | 344J16_region_M_1_8102_14_{—} | | | |
| | fw | | | |
| 4593 | 344J16_region_M_1_8102_14_{—} | | | |
| | rv | | | |

The information in Table 1 serves to identify the SSR-containing loci and primers where

“Seq Num” is SEQ ID NO. for the sequence listing.

“Seq Id” is a name which provides mapping data. For instance, for SEQ ID NO:2, the elements of the Seq Id are “240G09_region_A2_{—}1_{—}1076_{—}51” where “240G09” is an arbitrary contig name, “region_A2” indicates that the SSR is in linkage group A2, the numeral “1” indicates the sequential order of the contigs in the region, the numeral “1076” indicates that the starting nucleotide base in the contig for the SSR, and the numeral “51” indicates the nucleotide length of the SSR. For the primers of SEQ ID NO: 1532 through SEQ ID NO:4593 the Seq Id corresponds to the Seq Id of the cognate locus followed by “fw” or “rv” for the forward or reverse primer, respectively, for amplifying a locus.

“Repeat Unit”, “Repeat Times” and “Distance from Previous Marker” describe the SSR and its physical location. For SEQ ID NO:2, the repeat unit is AT, repeated 26 times and beginning 912 bases from the start of the previous SSR in the contig.

There is some discrepancy in numeration of markers because the marker selecting software adds common bases which may not fit in a complete repeat unit. A further peculiarity of the marker selecting software is an inability to recognize SNPs in an SSR. For example see the marker of SEQ ID NO: 24, which to the eye is an ATATT repeat unit; but a SNP causes the repeat unit to be stated as “ATATTATATTATACTATATTA”. Known public SSR markers included in the SSRs are identified in Table 2.

### EXAMPLE 3

This example serves to illustrate mapping of the SSRs. Table 2 identifies a number of public SSR markers within the SSRs of Table 1 which are in linkage groups A2 and G. The location of these public SSR markers serves to locate the SSRs within regions of linkage groups A2 and G on genetic maps as illustrated in FIGS. 1 and 2.

No public SSR markers were identified as being within the SSR-containing regions of linkage groups M, A1 and H. However, an SSR of each of the regions of linkage groups M, A1 and H was mapped to adjacent public markers. Reference is made to the distance to adjacent public markers as described in Table 3.

TABLE 2

| Linkage Group | SSR SEQ ID NO: | Public Marker |
|---|---|---|
| A2 | 8 | Satt315 |
| A2 | 58 | Satt632 |
| A2 | 182 | Sat_162 |
| A2 | 472 | Satt424 |
| G | 560 | Satt275 |
| G | 658 | Satt163 |
| G | 798 | Sat_168 |
| G | 819 | Satt309 |
| G | 1063 | Sat_141 |
| G | 1065 | Sat_163 |
| G | 1181 | Satt610 |
| G | 1249 | Satt570 |
| G | 1303 | Satt235 |

TABLE 3

| | | Distance to |
|---|---|---|
| Linkage Group | Marker | next marker |
| M | SEQ ID NO: 1531 | 5.0 cm |
| | Satt636 | 6.2 |
| | Satt201 | 4.9 |
| | Sat_316 | — |
| A1 | Satt619 | 11.1 cm |
| | SEQ ID NO: 1403 | 22.3 |
| | Satt155 | — |
| H | Satt353 | 16.7 cm |
| | SEQ ID NO: 1476 | 14.9 |
| | SATT442 | — |

### EXAMPLE 4

This example illustrates the use of SSR markers in soybean QTL or gene association. The markers of this invention are useful in genotyping soybean lines for QTLs associated with soybean cyst nematode (SCN) resistance or susceptibility. SCN is a destructive pest of soybean resulting in high yield loss. Currently, the most cost effective control measures are crop rotation and the use of host plant resistance. While breeders have successfully developed SCN resistant soybean lines, breeding is both difficult and time consuming due to the complex and polygenic nature of resistance. The resistance is often race specific and does not provide stability over time due to changing SCN populations in the field. In addition, many of the resistant soybean varieties carry a significant yield penalty when grown in the absence of SCN.

Matson and Williams (Crop Sci. 5:447 (1965)) have reported a dominant SCN resistance locus, Rhg4, which is tightly linked to the ‘i’ locus on linkage group A2. In U.S. Pat. No. 5,491,081, incorporated herein by reference, Webb reports on the analysis of 328 recombinant inbreed lines (RIL) derived from a cross between soybean lines PI437654 and BSR101. Webb reported six QTLs associated with SCN resistance on linkage groups A2, C1, G, M, L25 and L26 and that the QTLs on linkage groups A2, C1, M, L25 and L26 act in a race specific manner. The QTL reported by Webb on linkage group A2 maps near the ‘i’ locus and is considered to be Rhg4 (U.S. Pat. No. 5,491,081). Webb concludes that only two loci on linkage groups A2 (Rhg4) and G (rhg1) explain the genetic variation to race 3.

Any soybean plant having an Rhg4 SCN resistant allele can be used in conjunction with the present invention. Soybeans with known Rhg4 SCN resistant alleles can be used. Such soybeans include, but are not limited to, PI548402 (Peking), PI437654 (Er-hej-jan), PI1438489 (Chiquita), PI507354 (Tokei 421), PI548655 (Forrest), PI548988 (Pickett), PI88788, PI404198 (Sun Huan Do), PI404166 (Krasnoaarmejkaja), Hartwig, Manokin, Doles, Dyer, and Custer. In a preferred aspect, the soybean plant having an Rhg4 SCN resistant allele is an Rhg4 haplotype 3 allele in a plant having either an rhg1 haplotype 2 or rhg1 haplotype 4 allele. Examples of soybeans with an Rhg4 haplotype 3 allele are PI548402 (Peking), PI88788, PI404198 (Sun huan do), PI438489 (Chiquita), PI437654 (Er-hejjan), PI404166 (Krasnoaarmejkaja), PI548655 (Forrest), PI548988 (Pickett), and PI507354 (Tokei 421). In addition, using the methods or agents of the present invention, soybeans and wild relatives of soybeans such as Glycine soja can be screened for the presence of Rhg4 SCN resistant alleles.

Table 4 below is a table showing single nucleotide polymorphisms (SNPs) for three haplotype sequences of Rhg4.

TABLE 4

| Identification | | | Phenotypes | | SSR Marker (SEQ ID NO:) | | |
|---|---|---|---|---|---|---|---|
| Hap | PI number | Line | SCN | Coat | 177 | 175 | 192 |
| 1 | — | A2069 | R | Yellow | 2 | 2 | 2 |
| 1 | — | A2869 | R | Yellow | 2 | 2 | 2 |
| 1 | — | A3244 | S | Yellow | 2 | 2 | 2 |
| 1 | PI87631 | Kindaizu | R | Yellow | 2 | 2 | 2 |
| 1 | PI548389 | Minsoy | S | Yellow | 2 | 2 | 2 |
| 1 | PI518664 | Hutcheson | S | Yellow | 2 | 2 | 2 |
| 1 | PI548658 | Lee 74 | S | Yellow | — | 2 | 2 |
| 2 | PI540556 | Jack | R | Yellow | 2 | 2 | 1 |
| 2 | PI360843 | Oshimashirome | R | yellow | — | — | — |
| 2 | PI423871 | Toyosuzu | R | yellow | — | — | — |
| 3 | PI548402 | Peking | R | black | 1 | 1 | 1 |
| 3 | PI88788 | — | R | black | 1 | 1 | 1 |
| 3 | PI404198 B | (Sun huan do) | R | black | 1 | 1 | 1 |
| 3 | PI438489 B | (Chiquita) | R | black | 1 | 1 | 1 |
| 3 | PI437654 | Er-hej-jan | R | black | 2 | 1 | 1 |
| 3 | PI404166 | Krasnoaarmejkaja | R | black | 1 | 1 | — |
| 3 | PI290136 | Noir | S | black | 1 | 1 | 1 |
| 3 | PI548655 | Forrest | R | yellow | 1 | 1 | 1 |
| 3 | PI548988 | Pickett | R | yellow | 1 | 1 | 1 |
| 3 | PI507354 | Tokei 421 | R | yellow | 1 | 1 | 1 |
| N/A | PI467312 | Cha-mo-shi-dou | R | GnBr | 1 | 1 | 1 |
| N/A | PI209332 | No. 4 | R | black | 2 | 2 | 2 |
| N/A | PI518672 | Will | S | yellow | 2 | 2 | 2 |
| N/A | PI548667 | Essex | S | yellow | 2 | 2 | 2 |

In Table 4, discrete haplotypes are designated 1 through 3. N/A refers to a haplotype that is not characterized. In Table 4, the Plant Introduction classification number is indicated in the “PI#” column. A dash indicates that no PI number is known or assigned for the line under investigation. The line from which the sequences are derived is indicated in the “line” column, with a dash indicating an unknown or unnamed line. The “Phenotypes.” columns of Table 4 indicate “SCN” resistance (R) to at least one race of SCN, or sensitivity (S) and “coat” color of a seed as either yellow, black, green/brown (GnBr), or unknown/unassigned (dash). At the I locus, black seeded varieties harbor the i allele for black or imperfect black seed coat; commercially preferred embodiments have a yellow coat. Three different SSR markers that occur within the loci of SEQ ID NOs: 175, 177 and 192 are listed under “SSR markers.” The allele of each marker occurring in a haplotype is indicated by a 1 or a 2, with a dash indicating that the information is not determined.

### EXAMPLE 5

This example illustrates the mapping of SSR markers. The genetic linkage of marker molecules of the present invention can be established on soybean linkage group A2 by a gene mapping model such as, without limitation, the flanking marker model reported by Lander and Botstein, Genetics, 121:185-199 (1989), and the interval mapping, based on maximum likelihood methods described by Lander and Botstein, Genetics, 121:185-199 (1989), and implemented in the software package MAPMAKER/QTL (Lincoln and Lander, Mapping Genes Controlling Quantitative Traits Using MAPMAKER/QTL, Whitehead Institute for Biomedical Research, Cambridge, Mass., (1990). Additional software includes Qgene, Version 2.23 (1996), Department of Plant Breeding and Biometry, 266 Emerson Hall, Cornell University, Ithaca, N.Y. Use of Qgenc software is one approach. The genetic linkage of SSR markers on linkage group A2 to the yield locus Sy5 are shown in Table 5. Soybean gene sequences found on linkage group A2 to be in genetic linkage with the Sy5 locus comprise the S-adenosyl-L-homocystein hydrolase (SAHH) gene, xyloglucan endotransglycosylase (XET1) gene, and the clhalcone synthase gene cluster.

TABLE 5

| | |
|---|---|
| Markers | Distance |
| Satt315 | 6.9 cM |
| SSR SEQ ID NO: 99 (Sy36) | 0.6 cM |
| XET1 gene | 0.3 cM |
| SSR SEQ ID NO: 203 | 0.1 cM |
| (SCNB187) | |
| SAHH gene | 0.1 cM |
| SSR SEQ ID NO: 228 | 0.1 cM |
| (SCNB188) | |
| SSR SEQ ID NO:253 | 0.1 cM |
| (SCNB190) | |
| SSR SEQ ID NO: 272 (Sy50) | 0.0 cM |
| Seed coat color | 1.1 cM |
| Sat_212 | 0.4 cM |
| Satt187 | 0.1 cM |
| Sat_215 | — |

### EXAMPLE 6

This example illustrates in more detail the use of markers of this invention in marker assisted breeding for the Sy5 yield QTL on soybean linkage group A2. DNA is extracted from healthy leaf of a young soybean plant. Aliquots of the DNA are PCR amplified using primer pairs of each of the following SSR-containing loci: SEQ ID NO: 99, SEQ ID NO:203, SEQ ID NO:228, SEQ ID NO:253 and SEQ ID NO: 272. Primers for the SSR markers are defined in Table 1. The PCR reaction products are scored by electrophoresis for the presence or absence of the bands on the appropriate molecular weights of SSR markers spanning the Sy5 yield QTL.

### EXAMPLE 7

This example illustrates the use of markers of this invention in breeding for a QTL. To facilitate the use of this exotic locus in improving yield of commercial cultivars the following procedure can be used. Briefly, a cross can be made with any of the progenies derived from the above described plants and derivatives thereof carrying an exotic locus with any potential cultivar that one wishes to improve. Using marker analysis a breeder can monitor the positive transfer of the exotic locus by checking the presence of the molecular marker band corresponding to markers associated with the exotic locus, i.e. the amplicon resulting from PCR amplification of DNA using marker primers. Then a series of backcrosses (up to BC₅) to the commercial cultivar (recurrent parent) can be made to recover most of the agronomic properties of the recurrent parent. Prior to each backcross step, the positive transfer of the exotic alleles has to be validated among backcross-derived progenies (BCnFn) (where n=generation) using molecular marker analysis as previously described. The number of backcrosses depends on the level of recurrent parent recovery which can also be facilitated by the use of markers evenly distributed throughout the genome.

Glycine max PI290136 having the desirable Sy5 yield locus and undesirable black seed coat trait is used as a donor parent (D) for crossing with elite soybean line H5050 (Hartz Seed, Stuttgart, Ark.) having the desirable yellow seed coat trait as the acceptor parent (A) following a protocol for isoline development for breaking linkage between Sy5 locus and black seed color. The elite, yellow seed coat line is crossed to a black seed coat donor parent carrying Sy5 QTL, producing F₁ plants which are heterozygous throughout the Sy5 region. The F₁ plants are back crossed producing BC₁F₁ plants which segregate at a 1:1 ratio for elite A line and donor parent alleles. The BC₁F₁ plants are genotyped with 2 SSR markers flanking Sy5 (e.g. SSR SEQ ID Nos: 99 and 272). Individuals that are heterozygous for both flanking markers and the black seed coat donor are selected. BC₂F₁ plants segregate 1:1 in the Sy5 region because all BC₁F₁ parents are heterozygous. The BC₂F₁ plants are genotyped with the same 2 SSR markers flanking Sy5 that are used in the BC₁F₁ to identify individuals that are heterozygous for both flanking markers. BC₂F₂ plants are genotyped with four SSR markers in the Sy5 region to identify plants which are heterozygous at all four marker loci. Self pollinated seed is harvested in bulk from the heterozygotes. The BC₂F₃ generation will segregate in a 1:2:1 ratio at the I locus (II:Ii:ii). Seed harvested from yellow plants, (II and li), segregates in a 1:2 ratio (II:Ii). F₃:₄ lines are planted from seed derived from yellow seed coat parents. Nonsegregating rows for seed color which arose from homozygous yellow parents are identified. Yellow seed coat parents segregate at 2:1 (Ii:II), hence, 1/3 of BC₂F₃.₄ rows will be uniformly yellow seed coat. BC₂F₃ plants are genotyped using flanking SSR markers. Desired BC₂F₃ plants carry one parental gamete throughout the Sy5 region and one recombinant gamete. BC₂F₃:₄ lines are desired that arose from BC₂F₃ individuals that are homozygous yellow seed coat and contain one parental gamete and one recombinant gamete close to the I locus. Homozygous yellow BC₂F₃ individuals are the result of randomly sampling two gametes.

Pollen from the F₁ progeny of that cross are then crossed back to the parent line to generate about 40 BC₁F₁ progeny. Each BC₁F₁ progeny is then grown and crossed again to the parent line to generate between 250 and 300 BC₂F₁ progeny. The BC₂F₁ progeny are grown and leaf samples are taken from each plant for subsequent DNA extraction and molecular marker genotyping. The BC₂F₁ plants are grown to maturity and genotyped with the molecular markers flanking the Sy5 locus. Nine BC₂F₁ heterozygote lines for both flanking markers are identified. The BC₂F₂ seeds are collected from each BC₂F₁ plant then bulked. The resulting seeds from each of BC₂F₁-derived progeny are used for yield field trials.

The yield field trial plots are laid out in a random split block design with a single replication, where blocks represent early, mid and late maturity groups to facilitate harvest. There are two-row 16-ft. plots, with the adapted parent, as a border row on each side. Seeding rate is eight seeds per foot. Cultural practices such as herbicide applications and fertilization are carried out following the recommendations for soybean. At harvest, only the test rows are harvested and seed yield is adjusted to 13% moisture content to get the dry yield for each line using the formula: Dry yield=Actual yield×(1-% moisture at harvest)/(1-0.13). Seed yield per plot is converted into yield in bushels per acre using the formula: Plot size/Acre=lb/Acre. For example, yield measured in lbs. from a 16-ft×5 ft plot is converted to bushels per acre by multiplying it with a factor of 9.075. In all cases, the average percent yield increase of the plants carrying the Sy5 yield QTL derived from PI290136 is statistically significant (Analysis of Variance) higher than that of the plants homozygous for the adapted alleles (Table 6).

TABLE 6

| Genotype | Mean (bu/Ac)¹ |
|---|---|
| | First year |
| Homozygous Sy5 QTL | 54.35 |
| Heterozygous Sy5 QTL | 53.47 |
| Sy5 QTL negative | 44.23 |
| | Second year |
| Homozygous Sy5 QTL | 38.25 |
| Heterozygous Sy5 QTL | 41.30 |
| Sy5 QTL negative | 31.69 |

¹Yield is measured as dry seed weight in bushels per acre.

We claim:

1. An SSR-containing soybean DNA locus which is useful for genotyping between at least two varieties of soybean and comprising genomic sequence adjacent to an SSR; wherein said genomic sequence is at least 90% identical to the sequence of a reference polynucleotide of at least 20 consecutive nucleotides which are adjacent to an SSR identified in Table 1 or the complement of such reference polynucleotide, but excluding the public SSR markers of SEQ ID NO:8 (Satt315), SEQ ID NO:58 (Satt632), SEQ ID NO: 182 (Sat_{—}162), SEQ ID NO:472 (Satt424), SEQ ID NO:560 (Satt275), SEQ ID NO:658 (Satt163), SEQ ID NO:798 (Sat 168), SEQ ID NO:819 (Satt309), SEQ ID NO:1063 (Sat_{—}141), SEQ ID NO:1065 (Sat 163), SEQ ID NO:1181 (Satt610), SEQ ID NO:1249 (Satt570) and SEQ ID NO:1303 (Satt235).

2. An SSR-containing soybean DNA locus according to claim 1 wherein said sequence of the locus is at least 95% identical to the sequence of the reference polynucleotide.

3. A data set of DNA sequences comprising up to a finite number of distinct sequences of loci according to claim 1 wherein said finite number is selected from the group consisting of 2, 5, 10, 25, 40, 75, 100, 500 and 1000.

4. A computer readable medium having recorded thereon a genetic or physical map of at least part of the soybean genome comprising map positions of two or more SSR-containing loci according to claim 1.

5. A computer readable medium according to claim 4 wherein said genetic or physical map for soybean comprises mapped SSR-containing loci for soybean chromosomes identified as linkage group G or A2.

6. A computer readable medium according to claim 4 wherein said map for soybean is as represented by Table 1.

7. An isolated nucleic acid molecule useful for detecting an SSR-containing locus in soybean DNA, wherein said nucleic acid molecule comprises at least 18 nucleotide bases, and wherein the sequence of said at least 18 nucleotide bases is at least 90 percent identical to a sequence of the same number of consecutive nucleotides in either strand of a segment of soybean DNA in a locus of claim 1 comprising said SSR-containing locus.

8. An isolated nucleic acid molecule according to claim 7 comprising at least 20 nucleotide bases.

9. A pair of isolated nucleic acid molecules useful for PCR amplification of a segment of soybean DNA comprising at least one SSR, wherein each nucleic acid molecule of said pair comprises at least 18 nucleotide bases and wherein the nucleotide sequence of one of said molecules is at least 90 percent identical to a sequence of the same number of consecutive nucleotides in one strand of a segment of soybean DNA in a locus of claim 1 comprising said polymorphism and the sequence of the other of said molecules is at least 90 percent identical to a sequence of the same number of consecutive nucleotides in the other strand of the segment of soybean DNA in said locus.

10. A pair of isolated nucleic acid molecules according to claim 9, wherein said first and second sequences flank an SSR in the locus according to claim 1 identified by SEQ ID NO:“n”, said pair comprising the sequences of SEQ ID NO: (1531+2n−1) and SEQ ID NO: (1531+2n), where “n” is an number between 1 and 1531.

11. A collection of at least up to a finite number of pairs of isolated nucleic acid molecules according to claim 10 wherein said finite number is selected from the group consisting of 2, 5, 10, 25, 40, 75, 100, 500 and 1000.

12. A method of finding polymorphisms in soybean DNA comprising comparing DNA sequence in at least two soybean lines wherein said sequence is selected by using a segment of a locus of claim 1.

13. A method according to claim 12 wherein said sequence is selected as being at least 80% identical to sequence of said locus.

14. A method of genotyping comprising assaying DNA or mRNA from tissue of at least one soybean line to identify the presence of a nucleic acid polymorphism linked to a locus of claim 1.

15. A method of genotyping according to claim 14 wherein said polymorphism is a mapped SSR-containing locus of claim 1.

16. A method according to claim 14 further comprising identifying one or more phenotypic traits for at least two soybean lines and determining associations between said traits and polymorphisms.

17. A method according to claim 14 wherein lines with complementary traits are identified and selected for breeding to introgress a phenotype.

18. A method according to claim 14 wherein said assaying employs sufficient nucleic acid molecules to identify the presence of at least up to a finite number distinct SSR-containing loci wherein said finite number is selected from the group consisting of 2, 5, 10, 25, 40, 75, 100, 500, 1000, 2000, 3000, 4000 and 5000.

19. A method of investigating a soybean allele comprising determining the presence of an SSR in the nucleic acid sequence of nucleic acid molecules isolated from one or more soybean plants wherein said SSR is linked to a locus of claim 1.

20. A method of mapping soybean genomic sequence comprising identifying the presence of a mapped SSR in said sequence, wherein said mapped SSR is linked to a locus of claim 1.

21. A method according to claim 20 wherein said mapped SSR is in a locus of claim 1.

22. A method according to claim 21 comprising identifying the presence of at least a finite number of said mapped SSRs, wherein said finite number is selected from the group consisting of 2, 5, 10, 25, 40, 75, 100, 500 and 1000.

23. A method of breeding soybean comprising selecting a soybean line having an SSR associated by linkage disequilibrium to a trait of interest wherein said SSR is linked to a locus of claim 1.

24. A method according to claim 23 comprising selecting a soybean line having at least a finite number of said mapped SSRs, wherein said finite number is selected from the group consisting of 2, 5, 10, 25, 40, 75, 100, 500 and 1000.

25. A method of associating a phenotype trait to a genotype in soybean comprising
(a) identifying a set of one or more distinct phenotypic traits characterizing said soybean plants,
(b) selecting tissue from at least two soybean plants having allelic DNA and assaying DNA or mRNA from said tissue to identify the presence or absence of a set of distinct SSR polymorphisms,
(c) identifying associations between said set of polymorphisms and said set of phenotypic traits,
wherein said set of polymorphisms comprises at least one SSR linked to a locus of claim 1.

26. A method of associating a phenotype trait to a genotype in soybean according to claim 25 wherein said set of polymorphisms comprises at least 10 SSRs linked to mapped SSRs of claim 1.

27. A method of associating a phenotype trait to a genotype in soybean according to claim 26 wherein said set of polymorphisms are linked to at least a finite number of said loci, wherein said finite number is selected from the group consisting of 2, 5, 10, 25, 40, 75, 100, 500 and 1000.

28. A method of associating a trait to a genotype in soybean according to claim 27 wherein the soybean plants are in a segregating population; wherein said DNA is allelic in a loci of a chromosome which confers a phenotypic effect on a trait of interest and wherein a polymorphism is located in said loci; and wherein the degree of association among said polymorphisms and between said polymorphisms and the traits permits determination of a linear order of the polymorphism and the trait loci.

29. A method according to clam 28 wherein at least 5 polymorphisms are linked to said loci permitting disequilibrium mapping of said loci.

30. A method identifying genes associated with a trait of interest comprising identifying linkage of at least one SSR polymorphism to said trait of interest, wherein said polymorphism is linked to a locus of claim 1, identifying a genomic clone containing said locus and identifying genes linked to said locus.

31. A method according to claim 30 further comprising using said association in marker assisted breeding

32. A method according to claim 30 further comprising using association in marker assisted selection.

33. A method comprising screening for a trait comprising:
(a) interrogating a collection of SSR polymorphism wherein said collection has an average density of less than 10 cM on a genetic map of soybean; and
(b) correlating the presence or absence of an SSR polymorphism within said collection with said trait.
wherein said SSR polymorphisms are linked to loci of claim 1.

34. A method of soybean breeding comprising:
(A) crossing an first soybean line with a second soybean line to produce a segregating population;
(B) screening the segregating population with a DNA molecular marker for a member plant having an allele derived from said first line, wherein the allele is associated with a QTL or gene of interest; and
(C) selecting for further crossing and selection a member plant having said allele;
wherein said DNA molecular marker is an SSR polymorphic locus of claim 1.
